(19) United States
(12) Patent Application Publication
(10) Pub. No.: US 2017/0216536 A1
(43) Pub. Date: Aug. 3, 2017

(54) NEEDLE WITH CUTTING BLADE

(71) Applicant: Scott Science, LLC, Rogersville, MO (US)

(72) Inventor: Wendell Scott, Rogersville, MO (US)

(21) Appl. No.: 15/420,650

(22) Filed: Jan. 31, 2017

Related U.S. Application Data
(60) Provisional application No. 62/289,540, filed on Feb. 1, 2016.

(51) Int. Cl. A61M 5/32; A61M 5/158

(52) U.S. Cl. CPC A61M 5/3286; A61M 5/158; A61M 2207/00

(57) ABSTRACT

An improved needle is described for use in injecting fluids into biologic tissues. The needle is configured to reduce damage to the biologic tissue due to insertion of the needle. The tip of the needle is provided with a cutting blade to cut a perforation to allow the insertion of the cannula of the needle with reduced damage to the biologic tissue. Methods for forming the improved needle are provided.

### CROSS REFERENCE TO RELATED APPLICATION

This application claims the benefit of priority of U.S. Provisional Application No. 62/289,540 filed Feb. 1, 2016, which is hereby incorporated by reference in its entirety.

### FIELD OF THE INVENTION

The needle with cutting blade described herein relates to needles for injecting fluids into or withdrawing fluid from patients with a hollow core needle. More specifically, the disclosed needle relates to needles with tips designed to minimize the damage to biologic tissue upon the insertion of the needle into the patient. The disclosed needle improves the overall outcomes by reducing negative consequences of needle insertion such as leakage from perforated tissue or leakage from around a device inserted through the perforation. The disclosed needles reduce the coring, tearing or other damage to biologic tissue that may occur using a typical hollow needle. The disclosed needle also reduces the entry pressure necessary to penetrate biologic tissue, including without limitation eye, skin, organs, vessels, and neurologic tissues. This innovative needle is particularly well suited for injection into or withdrawal of liquids or gas from the eye.

### DESCRIPTION OF THE RELATED ART

Some needles with beveled cutting edges have been disclosed, such as that described in U.S. Pat. No. 7,070,583, U.S. Pat. No. 6,009,933, and U.S. Pat. No. 3,308,822. The needles described therein provide for cutting edges disposed at the edge of the needle cannula, not transecting the needle cannula or disposed along or parallel to the longitudinal axis of the needle cannula. The previously described needles cause coring and other damage to the biologic tissue. Moreover, when withdrawn the opening caused by the needle will leak due to the coring effect on tissue.

### SUMMARY OF THE INVENTION

In some embodiments, the invention comprises a needle for use with biologic tissue. The needle has a cannula with an anterior end (the point for insertion into biologic tissue), a posterior end (the end attached to the pump or syringe), and a lumen that extends along the longitudinal axis of the cannula. This embodiment of the needle has a lumen face at the anterior end of the cannula. The lumen face extends at an angle to the longitudinal axis of the cannula to reduce the impact of needle insertion on the biologic tissue. A blade extends from the anterior end of the lumen face. The lumen opens through an aperture in the lumen face. The blade has a planar surface that is approximately parallel to the longitudinal axis of the cannula. The blade has a cutting edge at the anterior end of the blade, which cuts a perforation in the biologic tissue as the needle is inserted into the tissue. This cutting edge cleanly divides tissue to create a generally linear opening through which the needle cannula passes thereby eliminating the common round core created by most needles. A linear “cut” is known to heal much faster than a non-linear opening or tear through the tissue. Moreover, significantly less pressure against the tissue is required to form a linear cut as opposed to other geometric openings.

In some embodiments of the needle, the maximum width of the blade is greater than the diameter of the cannula.

In some embodiments, the needle has a back face opposite to the lumen face that extends at an angle to the longitudinal axis of the cannula. The back face and the lumen face converge at their anterior ends where the cutting blade begins.

In some embodiments of the needle, the cutting edge of the blade is beveled to create a sharper cutting tool. The bevel may slope toward the upper or lower surfaces of the blade, or the edge may be beveled toward both upper and lower surfaces. In some embodiments of the needle, the thickness of the blade reduces toward its cutting edge.

In some embodiments of the needle, the blade has a groove on the upper surface of the blade with the groove running parallel to the longitudinal axis of the needle. In some embodiments of the needle, the cutting edge of the blade has more than one cutting segment. The cutting segments may form a point near the longitudinal axis of the needle shaft. In some embodiments of the needle, the surface of the blade is coincident with the longitudinal axis of the cannula so that the blade is centered over the shaft of the needle.

In some embodiments, the cannula of the needle is not uniformly circular in cross-section along its entire length. In some embodiments, the cannula includes a transition section adjacent to the lumen face. In some embodiments, the transition section changes from a circular cross-section adjacent to the remainder of the cannula to a non-circular at its anterior end near the lumen face. In some embodiments of the needle, the non-circular cross-section of the transition section of the cannula is an elliptical cross-section or an oval cross-section. In other embodiments, the cannula of the needle has a polygonal cross-section. In other embodiments of the needle, the lumen has a polygonal cross-section.

Some embodiments of the needle are manufactured by a method that includes a number of steps as described herein. The cannula (or shaft) of the needle may be cut at an angle to the longitudinal axis of the cannula to form an angled face. The lumen within the cannula opens through an aperture on the angled face. A portion of the angled face may then be flattened to form the blade. The portion of the angled face that is flattened extends from the tip of the needle to a point on the angled face between the tip and the lumen aperture. The anterior end of the blade is then cut or ground to form a cutting edge.

A portion of the cannula is bent toward the angled face to the approximate center of the posterior end of the blade over the center of the cannula of the needle. In some embodiments, the entire length of the shaft from the posterior end of the angled face to the tip is bent toward the angled face. The blade is bent away from the lumen face so that it extends approximately parallel to the longitudinal axis of the cannula.

Other embodiments of the needle are manufactured by a method that includes a number of steps as described herein. The cannula (or shaft) of the needle may be compressed to flatten the linear distal end of the cannula, resulting in a flattened linear distal end that is disposed approximately along the longitudinal axis of the cannula. The distal edge is then sharpened or cut to create a cutting blade. In the process, one or two apertures, inferior and/or superior relative to the needle edge, are created.

In some embodiments of the needle, additional apertures into the lumen may be created by laser or other means. These may be single or multiple, and may be at any angle or position relative to the distal end of the needle.

In some embodiments of the needle, the apertures are placed in a position and/or angle that create directionality for fluid, gas, or solid matter passing through the lumen of the needle.

In some embodiments of the needle, the edge of the aperture in the lumen face is dulled by any suitable means, including acid wash, polishing, laser cutting or ablation. In some cases, the dulling is performed by applying an acid wash to the needle prior to sharpening the blade edges.

In some versions of the method, the cutting edge of the blade is beveled to create a sharper cutting edge.

In other versions of the method, a groove is formed in the upper surface of the blade. The groove extends from the posterior edge of the blade to the tip of the needle

The foregoing steps for creating the claimed needle may be performed in other orders, and may use other technologies to form and create the needle.

### BRIEF DESCRIPTION OF THE DRAWINGS

FIG. 1 is a partial perspective view of an embodiment of the cutting blade end of the inventive.

FIG. 2A is a partial side plan view of an embodiment of the needle with cutting blade.

FIG. 2B is an end view of an embodiment of the needle with cutting blade.

FIG. 2C is a top view of an embodiment of the needle with cutting blade.

FIG. 3 is a perspective view of a second embodiment of the needle with cutting blade.

FIG. 4A is a side view of a second embodiment of the needle with cutting blade.

FIG. 4B is a end view of a second embodiment of the needle with cutting blade.

FIG. 4C is a top plan view of a second embodiment of the needle with cutting blade.

FIG. 5 is a perspective view of a third embodiment of the needle with cutting blade.

FIG. 6A is a side cross-sectional view of a third embodiment of the needle with cutting blade.

FIG. 6B is an end view of a third embodiment of the needle with cutting blade.

### DETAILED DESCRIPTION

When referring to the needle with cutting blade, posterior refers to the portions of the device closer to the syringe or pump, and anterior refers to portions of the device closer to the tip of the needle. The longitudinal axis of the shaft or cannula refers to the longitudinal axis of the polyhedron or cylinder formed by the shaft, which axis extends from the posterior end of the needle to the anterior end of the needle. In some cases the lumen 104 disposed in the shaft 102 is disposed along the longitudinal axis and in other cases it is offset from and substantially parallel to the longitudinal axis.

In some embodiments the needle may be formed with a circular, oval, elliptical, or polygonal cross-section. In some embodiments the cross-section of the cannula may vary along its length. The needles may be forged in such a shape or manipulated after initial manufacture to have all or a portion of the needle of a non-circular cross-section. The manipulation may include grinding to remove material, hammering or flattening, or other types of machining technologies. The needle cross-section may be selected to minimize the resistance to its insertion through a flat incision formed by the cutting blade as described below.

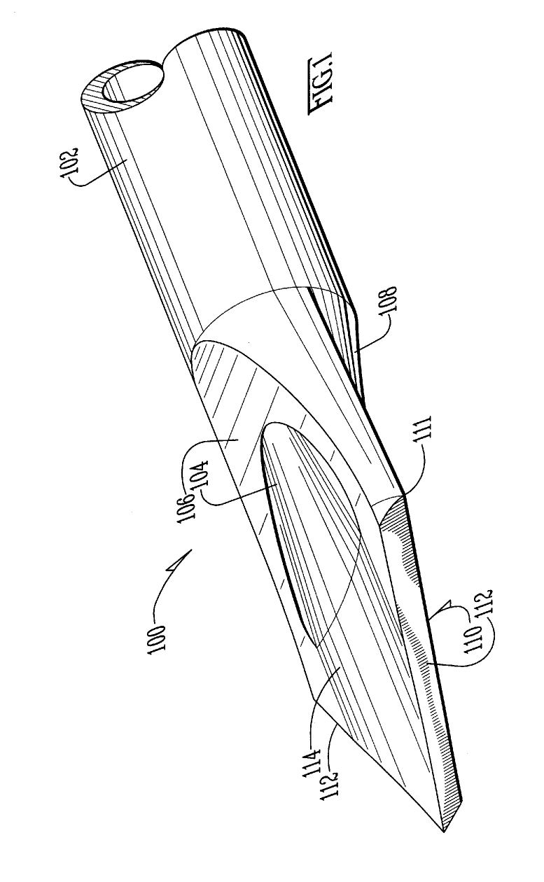

Referring now to FIG. 1, a perspective view of an embodiment of the needle with cutting blade is depicted. The needle 100 comprises a cannula or shaft 102 with an interior lumen 104 extending along the length or longitudinal axis of the cannula 102 for delivering fluid from a reservoir in communication with the lumen into the biologic tissue of a patient after the needle has been inserted therein.

When using a needle with a standard tip, the pressure of the biologic tissue is increased on all sides of the lumen of the cannula as the introduction of the needle itself displaces the tissue and may cause the removal of a core of biologic tissue in the lumen of the cannula, sometimes referred to as coring. The cutting blade of the needle reduces or eliminates coring by placing the cutting blade across the lumen of the needle as described below. This configuration may reduce the pressure on the biologic tissue from the edges of the lumen by transferring some of that pressure to a back face, described below, that does not have an opening to the lumen and thus cannot core the biologic tissue. In addition, the elastic nature of biologic tissue allows stretching to occur around the needle. With the standard needle the diameter of the incision is not wide enough to permit passage of the needle without undue pressure on the internal lumen and the entry wound. This tissue pressure results in coring from the internal lumen and in irregular tearing of tissue extending from the outer walls of the needle. This results in an entry wound than is compromised by removal of tissue from coring and unpredictable enlargement by tearing of the tissue, both of which contribute to wound leakage and susceptibility to bacteria entry. As described below, the increased width of the cutting blade allows the needle to pass through the tissue by optimizing the ability of the tissue to stretch around the needle without causing coring or tearing of the tissue. The entry wound is thus in better opposition when the needle is removed, allowing it to seal, preventing leakage and bacteria penetration.

The anterior end of the shaft 102 has been modified from conventional geometry to form a cutting blade 110 to reduce damage to the biologic tissues of the patient caused by insertion of the needle. The cutting blade 110 is fashioned from the end of the shaft 102 so that the cutting blade 110 transects the cross-section of the cannula 102. In other words the cutting blade 110 is disposed generally across the lumen of the cannula 102, not tangent to or at the surface of the cannula 102. In some embodiments, the cutting blade 110 is a planar surface which width transects the cross-section of the cannula 102 and is parallel to the longitudinal axis of the cannula 102. In some embodiments the planar surface of the cutting blade contains or is coincident with the central longitudinal axis of shaft 102 and lumen 104. Positioning the cutting blade so that it generally transects the cross-section of the cannula 102 reduces damage caused by the shaft 102 as it enters the hole or slit in the biologic tissue created by the cutting blade 110. In some embodiments of the needle, the cutting blade has one planar surface that is parallel to the longitudinal axis of the cannula 102 and a second back surface that is disposed at an angle to the planar surface.

In some embodiments the width of the cutting blade 110 is greater than the diameter of the cannula, and the ends of the cutting blade 110 extend past the outer surfaces of the cannula 102. The wider linear cut made by the cutting blade 110 allows tissue adjacent the cut to stretch around the needle with fewer unintended tissue tears or trauma.

In the embodiment depicted in FIG. 1, the final configuration of the needle includes a cutting blade 110, and a lumen face 106 disposed at an angle to the longitudinal axis of the shaft 102. Lumen 104 is open to an aperture in lumen face 106 for delivering fluid into the patient's tissues. The lumen face 106 is created by cutting, grinding, stamping or otherwise creating a face in shaft 102 that is at an angle to the longitudinal axis of the shaft 102. In some embodiments, the lumen face comprises the edges of the cannula after the cannula has been cut or ground to an angle.

The outside surface of the cannula that is bent to locate the cutting blade 110 across the lumen creates a back face 108 that receives some of the pressure of the biologic tissue during needle insertion, but since there is no aperture to the lumen, no coring or damage to biologic tissue occurs on back face 108. The disposition of the cutting blade 110 with respect to the lumen alters the amount of tissue pressure on the edges of the lumen 104. For example, if the cutting blade 110 is disposed through the central axis of the lumen, about half of the pressure of the biologic tissue is applied to the lumen face 104 and half to the back face 108. Since the back face 108 does not have an aperture into the lumen and no edges to cut the tissue, the pressure on back face 108 reduces the overall damage to the biologic tissue. Various embodiments may dispose the cutting blade 110 more toward the lumen face 106 or the back face 108.

In some embodiments, the angle between the lumen face and the longitudinal axis of shaft 102 is an acute angle. In some embodiments, the angle between the lumen face 106 and the longitudinal axis of shaft 102 after the formation of back face 108 by bending the needle is an acute angle. In some embodiments, an initially straight shaft 102 is cut or ground to form the angled face. A portion of the angled face posterior to the cutting blade 110 comprises lumen face 106.

In some embodiments of the needle, the edge of the aperture in lumen face 106 is dulled to reduce or prevent coring or cutting of biologic tissue by the edge of the aperture. The dulling may be performed by applying an acid wash to the needle after cutting it to form the angled face, or other suitable means of dulling the edge such as laser cutting or ablation.

In some embodiments, the cutting blade 110 is formed from the end of the angled face by flattening an anterior portion of the angled face. This portion of the angled face includes the area forming blade 110 and a portion of lumen face 106 to create a transition from the round shaft 102 to blade 110. In some embodiments, the flattening of the angled face creates a widened cross-axis shoulder 111 that is wider than the diameter of shaft 102. In some embodiments, the width of shoulder 111 is half of the circumference of shaft 102, corresponding to flattening the angled face where it is half way through the cylinder of the shaft 102. The blade 110 is shaped from the portion of the angled face anterior to cross-axis shoulder 111. In some embodiments, the width of blade 110 is wider than the diameter of shaft 102 to create a linear perforation or cut in the patient's biologic tissue that is better able to accommodate the circumference of the shaft 102 without tearing at the ends of the cut.

In some embodiments, the widest point of cutting blade 110 is selected to minimize the amount of resistance to and the amount of damaged caused by the insertion of the cannula 102 into the perforation created by cutting blade 110. In some embodiments the widest point of cutting blade 110 is at cross-axis shoulder 111, while in other embodiments the widest point of blade 110 is disposed at another point of the blade 110.

For embodiments created by flattening the angled face of the needle to form the cutting blade, the width of shoulder 111 may be adjusted by selecting the location of the shoulder 111 along the surface of the angled face. Forming shoulder 111 closer to the posterior end of the angled face will create a wider shoulder 111 and blade 110 as a result of the flattening of the cannula 102 at the location of shoulder 111. Forming shoulder 111 closer to the anterior end of the angled face will create a narrower shoulder 111 and blade 110.

Positioning the shoulder 111 at the point where the angled face is halfway through cannula 102 will result in a shoulder 111 having a width that is equal to half of the circumference of the cannula 102. Moving the shoulder 111 in a posterior direction will result in a shoulder 111 that is wider but with a more abrupt transition from blade 110 to cannula 102, thus potentially causing more damage to biologic tissue upon insertion. Moving the shoulder 111 in an anterior direction from that half circumference point will result in a shoulder 111 that is narrower with a longer transition, though the narrow width of the blade 110 may create a perforation in the biologic tissue that is insufficient to receive the cannula 102 resulting in tearing of the biologic tissue. The optimal width of shoulder 111 and cutting blade 110 may vary in differing applications.

As described above, the final configuration of the needle includes the disposition of the cutting blade 110 transecting the cross-section of the cannula 102. In order to dispose the end of the shaft 102 in the desired position, the shaft may be bent toward lumen face 106. The bending of shaft 102 creates back face 108 on the opposite side of shaft 102 from lumen face 106. In some embodiments, additional material may be added to back face 108 to strengthen it.

The blade 110 may also be angled toward the back face 108 to bring blade 110 into a parallel alignment with the longitudinal axis of the shaft 102. Blade 110 transects the cross-section of shaft 102 and may be substantially coincident with the longitudinal axis thereof, or it may be substantially parallel to but not coincident with the longitudinal axis. In some embodiments, the longitudinal axis of the shaft 102 is disposed substantially in the center of the shaft 102.

The anterior edge 112 of blade 110 may be configured with a variety of blade shapes. In the depicted embodiments, blade 110 has an “arrowhead” shape with two cutting segments that meet at a point within the longitudinal cross-section of cannula 102. In some embodiments, the angle of the “arrowhead” shape subtends an arc of approximately 66 degrees. In the depicted embodiment, the two cutting segments that comprise edge 112 are symmetrical and intersect at the center of blade 110, though in other embodiments they may intersect closer to one side of blade 110. In some symmetrical embodiments, the cutting segments of edge 112 subtend an arc of approximately 33 degrees on each side of the longitudinal axis of the shaft 102.

In other embodiments, there may be only one cutting segment on edge 112 of blade 110, or more than two cutting segments of edge 112. For example, an embodiment may incorporate a cutting edge that extends across the entire blade 100 with one end of the cutting edge disposed anterior to the other end, creating an angled cutting edge. In various embodiments, the cutting segments of edge 112 may be asymmetrical or may intersect with other cutting segments of edge 112 away from the longitudinal axis of the shaft 102 or the center of the blade 110.

In some embodiments, cutting edge 112 is sharpened to further reduce the entry pressure and damage to the biologic tissue caused by its insertion. In some embodiments, the sharpening comprises a bevel of the face of cutting edge 112 with respect to blade 110 to create a leading edge and a trailing edge of cutting edge 112. In some embodiments, the trailing edge is the upper edge of the cutting blade 110. In this context upper means on the same side of the cutting blade 110 as the lumen face 106. In some embodiments, the trailing edge is the lower edge of the cutting blade 110. In this context lower means on the same side of the cutting blade 110 as the back face 108. In other embodiments, the cutting edge 112 is beveled in upper and lower directions, resulting in a leading edge disposed between the upper and lower edges of cutting edge 112. Various different blade edge configurations and sharpening methods may be preferred in different applications.

In some embodiments, a groove 114 may be provided on the upper surface of blade 110. The groove 114 extends from the lumen face 106 toward the edge 112. The groove 114 accommodates some volume of soft biologic tissue displaced by the upper surface of cutting blade 110. The groove 114 may reduce pressure on the biologic tissue and secondary coring of the biologic tissue by the aperture in lumen face 106. In some embodiments the groove is added to the cutting blade 110, and in some embodiments, the groove 114 is a formed by the remainder of the surface of lumen 104 when the cannula 102 is cut to form blade 110.

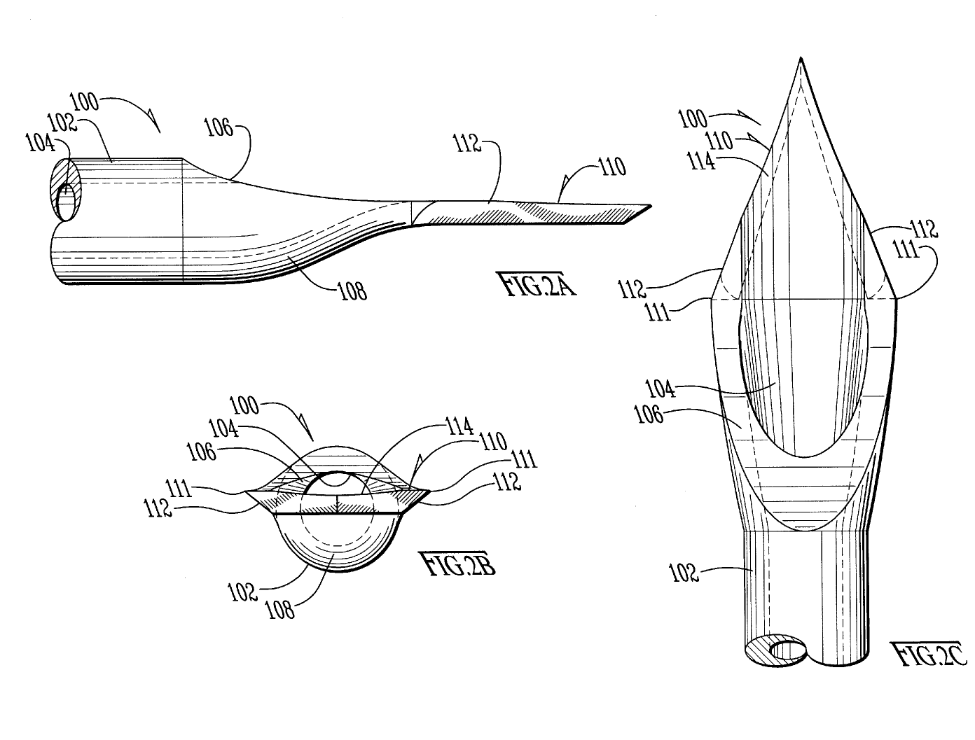

Referring now to FIGS. 2A, 2B, and 2C, side plan, end, and top views, respectively, of an embodiment of the needle with cutting blade are depicted. The description in relation to FIG. 1 applies equally to the embodiment depicted in these figures.

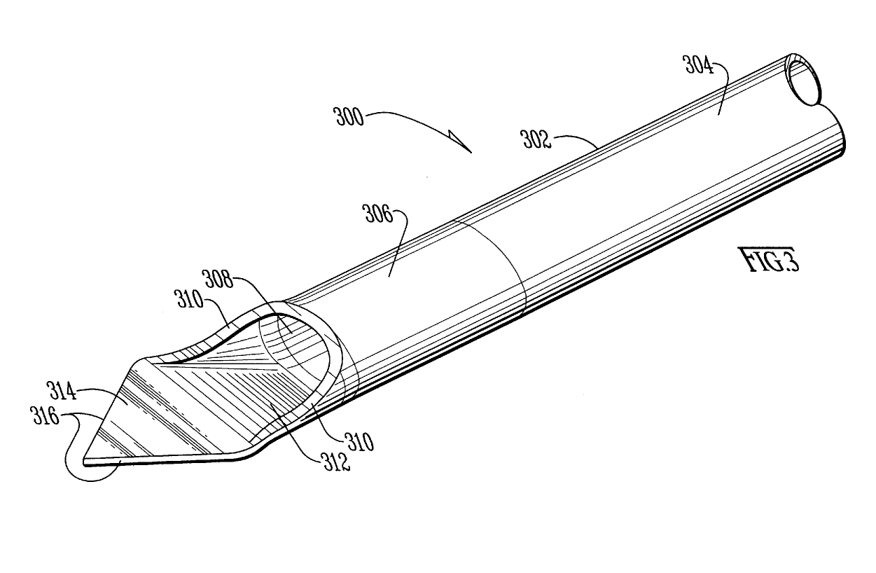

Referring now to FIG. 3, a perspective view of a second embodiment of the needle with cutting blade is depicted. In this embodiment of the needle with cutting blade, the needle 300 has a shaft 302 with a circular cross-section that transitions to an elliptical or oval cross-section prior to its termination in the cutting blade. The oval or elliptical cross-section may result in reduced resistance to the insertion of the needle 100 through the perforation created by blade 314 and thus to less damage to biologic tissue. If the modified shape of the cannula 302 also comprises a modification to the cross-section of lumen 308, there may be a resulting increase in resistance to fluid flow through lumen 308. Depending on the application for the needle 300, the optimal shape of the transition segment 304 may vary based on the type of biologic tissue and the type of fluid to be injected through lumen 308.

In some embodiments, the shape of the cannula 302 may be modified by grinding the outer surface of the cannula 302 without modifying the shape of the lumen 308.

In the depicted embodiment circular cross-section shaft 302 transitions in section 304 to section 306 with an elliptical or oval cross-section. Section 306 is oriented so that the major axis of the ellipsis or oval is parallel to the edge or edges 316 of blade 314. In the depicted embodiment, section 306 has an elliptical cross-section. The elliptical or oval shape of the section 306 of the shaft allows the shaft to cause less damage to the biologic tissue into which it is inserted because the major (longer) axis of section 306 is parallel to the cutting blade 314.

Similarly to the embodiment described in relation to FIGS. 1 and 2A-2C, the needle shaft is cut to create lumen face 310 and bent and flattened to create back face 312 and blade 314. Cutting edges 316 may be configured as those described in relation to FIG. 1.

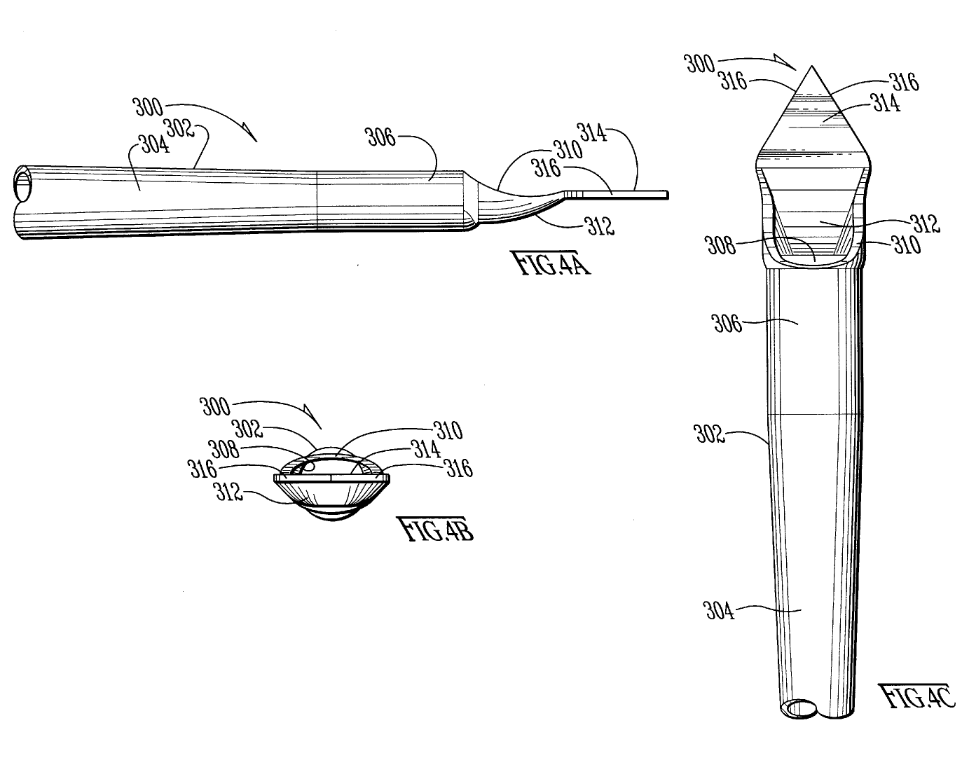

Referring now to FIGS. 4A, 4B, and 4C, side, end, and top views, respectively, of the second embodiment of the needle with cutting blade are depicted. The embodiment shown in FIGS. 4A, 4B, and 4C is described in detail with reference to FIG. 3, and the description thereof applies equally to the later figures.

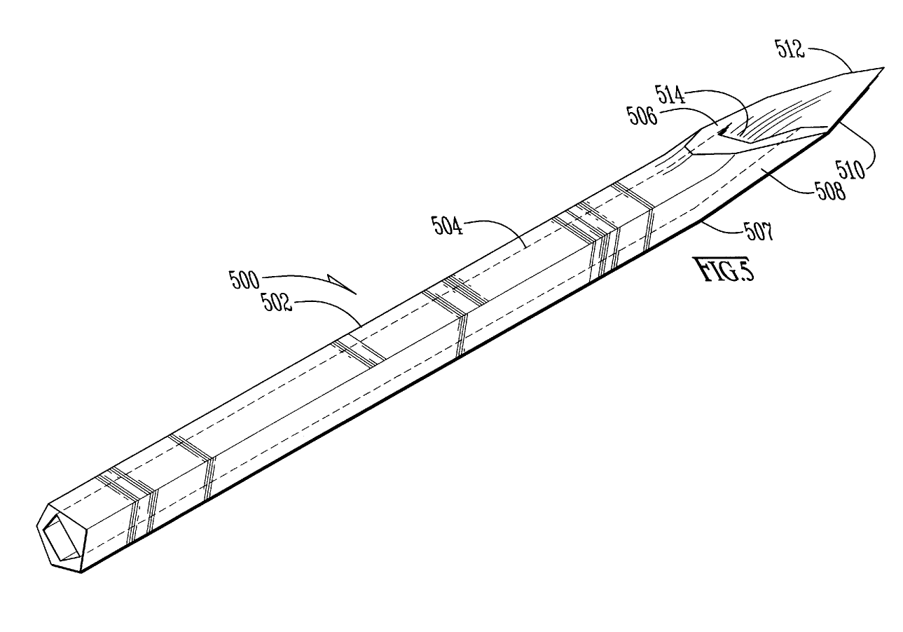

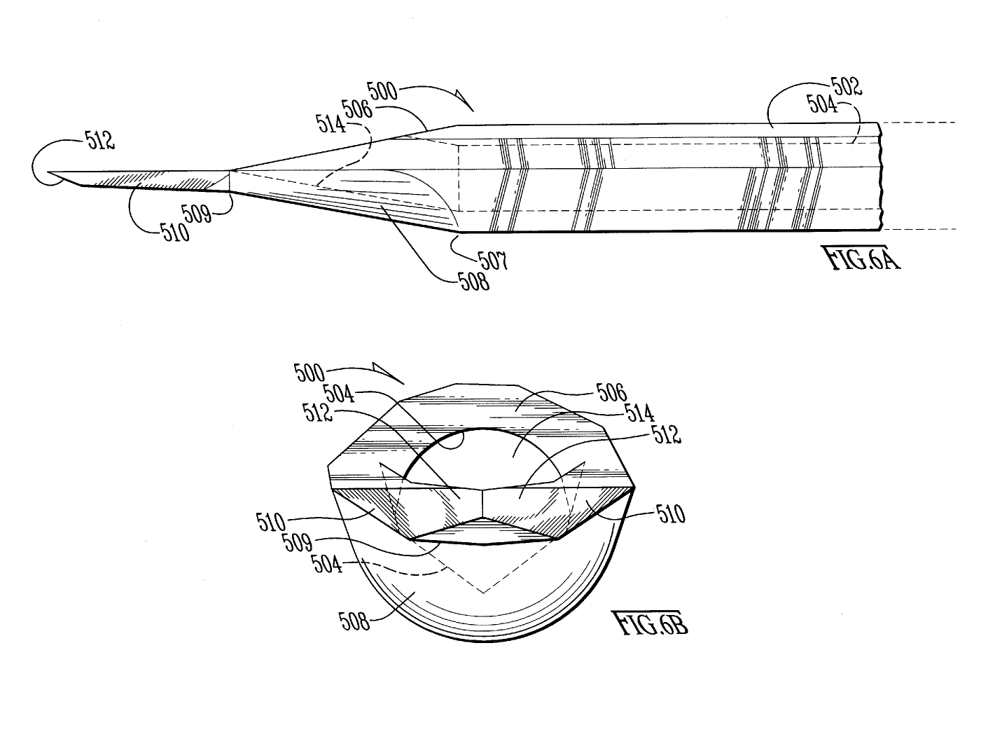

Referring now to FIGS. 5, 6A, and 6B, perspective, side cross-sectional and end views, respectively, of a third embodiment of the needle with cutting blade are depicted. The shaft or cannula 502 of needle 500 has a polygonal cross-section. In the embodiment depicted in FIG. 5, the shaft 502 has a pentagonal cross-section, but in other embodiments the shaft 502 may have cross-sections of other types of polygons.

The shaft 502 has a lumen 504 extending along the longitudinal axis of the shaft 502. In the depicted third embodiment, the lumen 504 has a polygonal cross-section. In the embodiment depicted in FIG. 5, the lumen 504 has a quadrilateral cross-section, though in other embodiments the lumen 504 may have cross-sections of other types of polygons.

In the depicted third embodiment, lumen face 506 has lumen aperture 514 in communication with lumen 505. Back face 508 extends at an angle to the longitudinal axis of cannula 502 and converges with lumen face 506. Cutting blade 510 extends from the anterior end of the lumen face 506 and back face 508.

In the depicted third embodiment of the needle with cutting blade, the lumen face 506 and back face 508 are disposed at angles to the longitudinal axis of shaft 502, and approach the longitudinal axis toward the anterior end of each face 506 and 508.

In the depicted embodiment, the lower surface of cutting blade 510 is beveled so that the thickness of blade 510 decreases toward its anterior end. In other embodiments, the upper surface may be beveled or both the upper and lower surfaces may be beveled along the length of blade 512. Blade edge 512 extends long the anterior end of blade 510. In this embodiment, edge 512 is beveled to create a sharp cutting edge coincident with the upper surface of the blade 512. In other embodiments, the sharp cutting edge may be coincident with the lower surface of blade 512 or disposed between the upper and lower surfaces of blade 512.

In various embodiments, the needle may be manufactured using a typical cannula 502 and cutting or grinding it to create an angled face extending at an angle to the longitudinal axis of the cannula 512 and through its thickness. The posterior portion of the angled face comprises the lumen face 506. The cannula 502 is then bent at shoulder 507 towards lumen face 506.

Then the angled face is flattened beginning at shoulder 509 and extending to the anterior tip of the needle forming blade 510. The blade 510 is then bent back toward the back face 508 until substantially parallel to the longitudinal axis of cannula 502.

The tip of the needle may then be cut or ground to form a desired blade shape and to form cutting edges on the anterior end of the blade. The blade 510 may be ground or cut to for a desired blade shape or to sharpen the edges 512.

Additional bending, grinding or addition of material to the needle may take place as part of the prior manufacturing process. Other manufacturing techniques may be utilized to create the final configuration of the needle.

In some embodiments of the needle with cutting blade, a shaft with a polygonal cross-section may have a circular, oval or elliptical lumen. In other embodiments, the shaft may have a cross-section that is circular, oval, or elliptical and the lumen may be entirely or partially polygonal. In other embodiments, the shaft or lumen of the needle may have a cross-section that is partially composed of curved sections and partially composed of straight sections. In some embodiments, the cross-section of the shaft or the lumen may be non-uniform along the length of the needle.

In some embodiments, the needle is manufactured from a standard needle cannula. The linear distal end of the cannula is compressed to form a flattened end that is disposed approximately along the center axis of the cannula. The compression of the cannula closes the lumen within the cannula. The flattened distal end is shaped and sharpened to create the desired cutting blade.

Either, before, after or during the sharpening of the distal end of the needle, one or more apertures are cut in the cannula to open to the lumen anterior to the compressed portion of the cannula. The apertures may be disposed superior, inferior or both, with respect to the flattened blade. In some embodiments, the apertures may be disposed at any angle or orientation with respect to the flattened blade. The apertures may be laser cut, or otherwise created to allow fluids to escape from the lumen during use of the needle. In some embodiments, the locations of the apertures are selected to cause the escaping fluid or gas to be directed in a desired orientation with respect to the cutting blade. The edges of the apertures may be dulled by acid wash, laser cutting or ablation, or other means, to reduce damage caused to biologic tissue due to contact with the edges of the apertures.

Many different arrangements of the various components depicted, as well as components not shown, are possible without departing from the spirit and scope of the present invention. Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

What is claimed is:

1. A needle for use with biologic tissue, the needle comprising:
a cannula having an anterior end, a posterior end, and a lumen extending along a longitudinal axis of the cannula from the anterior end to the posterior end of the cannula;
a lumen face at the anterior end of the cannula, the lumen face extending parallel or at an angle to the longitudinal axis of the cannula;
a blade extending from the anterior end of the lumen face, the blade comprising a substantially planar surface disposed substantially parallel to the longitudinal axis of the cannula, the blade having a cutting edge disposed at the anterior end of the blade;
wherein the lumen is in communication with an aperture disposed in the lumen face.

2. The needle of claim 1 wherein the maximum width of the blade is greater than the diameter of the cannula.

3. The needle of claim 1 further comprising a back face extending at an angle to the longitudinal axis of the cannula wherein the back face and the lumen face converge at their anterior ends.

4. The needle of claim 1 wherein the anterior edge of the blade is beveled.

5. The needle of claim 1 wherein the thickness of the blade reduces toward its anterior edge.

6. The needle of claim 1 wherein the blade further comprises a groove on the upper surface of the blade, the groove extending parallel to the longitudinal axis of the needle.

7. The needle of claim 1 wherein the planar surface of the blade is coincident with the longitudinal axis of the cannula.

8. The needle of claim 1 wherein the anterior edge of the blade has a plurality of cutting segments.

9. The needle of claim 1 wherein the cannula further comprises a transition section adjacent to the posterior end of the lumen face, the transition section having a cross-section that is circular at its posterior end and non-circular at its anterior end.

10. The needle of claim 9 wherein the non-circular cross-section at the anterior end of the transition section of the cannula is an elliptical cross-section.

11. The needle of claim 9 wherein the non-circular cross-section of the transition section of the cannula is an oval cross-section.

12. The needle of claim 1 wherein the cannula has a polygonal cross-section.

13. The needle of claim 1 wherein the lumen has a polygonal cross-section.

14. A method of manufacturing the needle of claim 1 from a cannula having a lumen, the method comprising the steps of:
cutting the needle at an angle to the longitudinal axis thereof to form an angled face, the lumen extending to the angled face through an aperture therein;
flattening a portion of the needle to form a blade, the portion extending from the anterior end of the needle to a point on the angled face;
cutting the anterior end of the blade to form a cutting edge;
bending a portion of the cannula toward the angled face, the portion extending from the posterior end of the angled face to the anterior end of the needle;
bending the blade away from the lumen face so that it is substantially parallel to the longitudinal axis of the cannula.

15. The method of claim 14 further comprising the step of dulling the edge of the aperture in the lumen face.

16. The method of claim 14 further comprising the step of beveling the cutting edge of the blade.

17. The method of claim 14 further comprising the step of forming a groove in the upper surface of the blade, the groove extending from the posterior edge of the blade to the anterior edge of the blade.

18. A method of manufacturing the needle of claim 1 from a cannula having a lumen, the method comprising the steps of:
compressing a portion of the distal end of cannula to form a cutting blade;
sharpening the distal end of the cutting blade to form a cutting edge;
cutting at least one aperture in the cannula posterior to the cutting blade.

19. The method of claim 18 further comprising the step of dulling the edge of the at least one aperture.

20. The method of claim 18 further comprising beveling the cutting edge of the cutting blade.

21. The method of claim 18 wherein the positions of the at least one apertures are selected to direct the flow of fluid or gas from the at least one aperture in a desired direction.
